(19) United States
(12) Patent Application Publication
(10) Pub. No.: US 2026/0013779 A1
(43) Pub. Date: Jan. 15, 2026

(54) PREDICTOR OF SEIZURE OUTCOME AFTER EPILEPSY SURGERY USING PERI-ICTAL SCALP EEG DATA

(71) Applicant: The Cleveland Clinic Foundation, Cleveland, OH (US)

(72) Inventors: Shehryar R. Sheikh, Cleveland, OH (US); Carl Saab, Moreland Hills, OH (US); Lara Jehi, Pepper Pike, OH (US)

(21) Appl. No.: 19/266,409

(22) Filed: Jul. 11, 2025

Related U.S. Application Data
(60) Provisional application No. 63/671,400, filed on Jul. 15, 2024.
(60) Provisional application No. 63/677,049, filed on Jul. 30, 2024.

(51) Int. Cl. A61B 5/374; A61B 5/00; A61B 5/291; G16H 10/60; G16H 50/20; G16H 50/70

(52) U.S. Cl. CPC A61B 5/374; A61B 5/291; A61B 5/4094; A61B 5/4848; A61B 5/7203; A61B 5/7235; A61B 5/7275; G16H 10/60; G16H 50/20; G16H 50/70

(57) ABSTRACT

A method of predicting a seizure occurrence or a surgical outcome includes monitoring a patient using an electroencephalography (EEG) system, including recording EEG data that indicates a seizure, and extracting a peri-ictal segment of the EEG data that includes a pre-ictal period immediately preceding the seizure, or a post-ictal period immediately following the seizure. The method also includes processing the peri-ictal segment, including generating an electrode-wise power spectral density (PSD) feature across a frequency band defined by at least one of a delta frequency range, a theta frequency range, an alpha frequency range, a beta frequency range, and a gamma frequency range. The method also includes inputting the PSD feature into a model, wherein the model predicts a seizure occurrence or a surgical outcome of the patient.

### CROSS REFERENCE TO RELATED APPLICATIONS

This application claims priority to and the benefit of U.S. Provisional Patent Application 63/671,400, filed on Jul. 15, 2024, entitled “A Predictor of Seizure Outcome After Epilepsy Surgery Using Peri-ictal Scalp EEG Data”, incorporated herein by reference in its entirety. This application also claims priority to and the benefit of U.S. Provisional Patent Application No. 63/677,049, filed on Jul. 30, 2024, entitled “A Predictor of Seizure Outcome After Epilepsy Surgery Using Peri-ictal Scalp EEG Data”, incorporated herein by reference in its entirety.

### BACKGROUND

Drug Resistant Epilepsy (DRE) afflicts over 20 million patients globally. Uncontrolled seizures in DRE result in devastating consequences for quality of life, increased healthcare costs, and increased mortality. Surgical brain resection is potentially curative for many patient but only about half achieve sustained seizure freedom. Every year, hundreds of patients assume the risks of brain resection (including visual field cuts, naming deficits, infections) while continuing to have debilitating seizures. The task of identifying DRE patients who are likely to experience recurrent seizures after resection is vital but challenging. Patients with drug resistant epilepsy are candidates for surgical brain resection as a treatment for seizures. However, only a subset of patients who undergo resection become seizure free postoperatively.

Seizure outcome prediction tools for epilepsy surgery are available but accuracy is modest. The field's best effort to date for developing a nomogram that predicts seizure outcome after epilepsy surgery uses clinical parameters and basic descriptors of the patient's brain imaging and scalp EEG to provide an estimated likelihood of seizure freedom. The predictive power of these nomograms is limited in large part because the patient-specific granularity in terms of physiological data that is key to robust outcome prediction is lost when the patient is described in simplified categories (e.g. instead of incorporating the full brain MRI for a specific patient, the model is only able to incorporate the binary value of ‘normal’ or ‘abnormal’). In other words, these models are not informed by raw patient data but rather by descriptors of that data (i.e. the model input is not a full MRI image, but rather a ‘yes/no’ description of whether the image is normal or abnormal). Thus much of the inter-patient variability in the patient data is lost in building the models.

Several efforts have attempted to incorporate more granular “raw” data into predictive frameworks, with most focusing on adding different imaging data as inputs (primarily preoperative structural MRI and brain PET). Although these studies added valuable insights to our understanding of epilepsy, their translation to clinical practice has been challenging for two main reasons. First, most were developed using modalities that are not part of routine presurgical evaluation, and are thus not easily translated into prediction model inputs for widespread utilization, such as diffusion tensor imaging, functional MRI, and intracranial EEG. Second, these studies have had an accuracy ceiling in the 70-80% range. No prior approaches have reached the elusive >90% accuracy range, where they would represent a promising investment of resources for development into a surgical outcomes prediction tool, and none have been developed to a stage where they can be incorporated into the presurgical decision-making workflow.

### SUMMARY

According to one aspect of the present disclosure, a method of predicting a seizure occurrence or a surgical outcome includes monitoring a patient using an electroencephalography (EEG) system, including recording EEG data that indicates a seizure, and extracting a peri-ictal segment of the EEG data that includes a pre-ictal period immediately preceding the seizure, or a post-ictal period immediately following the seizure. The method also includes processing the peri-ictal segment, including generating an electrode-wise power spectral density (PSD) feature across a frequency band defined by at least one of a delta frequency range, a theta frequency range, an alpha frequency range, a beta frequency range, and a gamma frequency range. The method also includes inputting the PSD feature into a model, where the model predicts a seizure occurrence or a surgical outcome of the patient.

According to another aspect of the present disclosure, a system for predicting a seizure occurrence or a surgical outcome includes an electroencephalography (EEG) system that monitors a patient and records EEG data indicative of a seizure. The system also includes at least one processor operatively coupled to the EEG system, where the at least one processor extracts a peri-ictal segment of the EEG data that includes a pre-ictal period immediately preceding the seizure or a post-ictal period immediately following the seizure, and generates a feature representative of the EEG data based on the peri-ictal segment. The at least one processor also integrates the feature with at least one clinical variable associated with the patient, the clinical variable indicating seizure frequency, history of generalized convulsions, cause of seizures, duration of epilepsy, gender, magnetic resonance imaging data or findings, EEG seizure localization, inter-ictal epileptiform discharge, a side of a surgery, an age at which the surgery was performed, or a follow-up period to the surgery. The processor also inputs the integrated feature and at least one clinical variable into a predictive model, where the model predicts a seizure occurrence or a surgical outcome of the patient.

### BRIEF DESCRIPTION OF THE DRAWINGS

FIG. 1 is a process flow diagram for developing a post-surgical seizure outcome prediction model.

FIG. 2A is a heatmap illustrating statistically significant differences in power spectral density across electrode-frequency pairs in pre-ictal and post-ictal epochs between surgical success and failure groups.

FIG. 2B is a heatmap illustrating statistically significant differences in power spectral density across electrode-frequency band pairs in pre-ictal and post-ictal epochs between surgical success and failure groups.

FIG. 3 is a series of plots comparing average power spectral densities across various frequency ranges for surgical success and failure groups.

FIG. 4 is a series of plots quantifying proportional representations of EEG frequency bands in surgical success and failure groups.

FIG. 5 is a receiver operating characteristic (ROC) curve comparing performance by various outcome prediction models.

FIG. 6A is a normalized confusion matrix for an EEG-augmented model.

FIG. 6B is a normalized confusion matrix for a non-EEG-augmented model.

FIG. 7A is a decision curve analysis (DCA) plot showing net benefit of using EEG-augmented versus non-EEG-augmented prediction models.

FIG. 7B is a plot indicating the net reduction in unnecessary surgeries achieved using EEG-augmented versus non-EEG-augmented models across the same threshold probability range.

FIG. 8 is a diagram of an EEG electrode placement scheme.

FIG. 9 is an exemplary operating environment of a system for predicting a seizure occurrence or a surgical outcome.

FIG. 10 is an illustration of a computer-readable medium or computer-readable device including processor-executable instructions configured to embody one or more of the provisions set forth herein, according to one aspect.

### DETAILED DESCRIPTION

It should be understood that the description and drawings herein are merely illustrative and that various modifications and changes can be made in the structures disclosed without departing from spirit and scope of the subject disclosure.

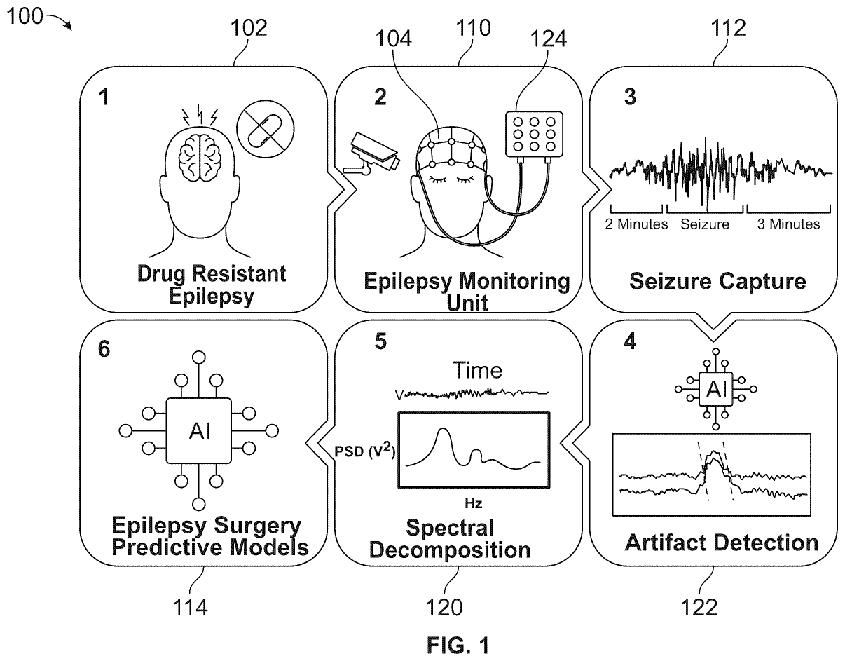

Referring now to the drawings, FIG. 1 depicts a process flow diagram that describes a method 100 for a post-surgical seizure outcome prediction model (such as a trained machine learning system), including generating and incorporating EEG data into a post-surgical seizure outcome prediction model. For simplicity, the method 100 will be described as a sequence of blocks, but the elements of the method 100 may be organized into different architectures, elements, stages, or processes.

As shown in FIG. 1, at block 102, the method 100 includes diagnosing a group of patients, including a patient 104, with drug resistant epilepsy (DRE). In an embodiment, the group of patients are diagnosed with drug resistant epilepsy of the temporal lobe after failing adequate trials of 2 anti-epileptic medications.

At block 110, the method 100 includes conducting an EEG study with the group of patients. In an embodiment, the EEG study may be an inpatient video-EEG study in which multiple seizures are recorded.

At block 112, the method 100 includes selecting EEG data generated from each patient in the group of patients for analysis. In this regard, one seizure may be chosen at random for each patient, and the EEG data indicates 2 minutes of immediately pre-ictal data and 3 minutes of immediately post-ictal data were captured for each patient.

At block 114, the method 100 includes applying a machine-learning based previously validated artifact detector to the raw EEG data time-series, where only artifact-free seconds of EEG data are utilized for subsequent analyses. At block 120, the method 100 includes converting the artifact-free EEG data from a time-series to a frequency domain using spectral decomposition, yielding an electrode-wise power spectral density.

At block 122, the method 100 includes training a post-surgical seizure outcome prediction model. In this regard, 75% of the dataset generated at block 120 is used for model building using an Auto-ML pipeline which recommends models based on 4-fold cross-validation. The remaining 25% of the dataset is used as a hold-out test set so that performance metrics based on unseen data could be reported.

In an embodiment, accuracy of the model trained at block 122 is within a clinical-translation range. In this regard, the model trained at block 122 has an area under the curve of 0.98. When tested on an “out of group” set of testing data that the model has never previously encountered, the accuracy may be >90%. Thus, the model trained at block 122 provides a relatively high degree of accuracy for post-surgical seizure outcome predictions.

The method 100, including training the model at block 122, may use raw peri-ictal EEG data, enhancing performance as compared to relatively preprocessed input data. In this regard, the model is augmented by the raw patient EEG data for enhanced accuracy. Furthermore, EEG data from immediately before and immediately after a patient experienced a seizure (i.e. peri-ictal EEG data) is employed. Specifically in one embodiment, the present model trained at block 122 uses 2 minutes of pre-seizure data and 3 minutes of post-seizure data to make its predictions.

The model trained at block 122 is a machine learning algorithm for predicting seizure control after temporal lobe resection using peri-ictal EEG. Notably, the window of time immediately before and after a seizure (“peri-ictal”) represents a unique brain state with implications for clinical outcome prediction. In this regard, an example dataset of 294 patients who underwent temporal lobe resection for seizures, machine learning classifiers were able to make accurate predictions of postoperative seizure outcome using 5 minutes of peri-ictal scalp EEG data that is part of universal presurgical evaluation (AUC 0.98, out-of-group testing accuracy >90%). Using that example dataset, a decision curve analysis (DCA) shows that compared to the prevalent clinical-variable based nomogram, use of the EEG-augmented approach could decrease the rate of unsuccessful brain resections by 20%.

The window of time immediately before and after a seizure (“peri-ictal” epoch) is uniquely informative for clinical outcome prediction. In a rat model test with spontaneous recurrent seizures and measuring intracranial local field potentials, seizures were reliably preceded by an increase in theta synchrony between the hippocampus and prefrontal cortex in the 2 minutes before a seizure with prompt dissipation post-ictally. Similarly, electrode recordings from the CA3 region of the hippocampus in a pilocarpine rat model of epilepsy showed that beginning minutes before a seizure, interneurons displayed progressive synchrony with oscillations in the theta, gamma, and finally ictal spiking frequencies. These pre-clinical studies support a model of ictogenesis where electrophysiological brain activity in the minutes immediately before and after the ictal event is quantitatively and qualitatively distinct. Based on these findings, the differences in functional connectivity that would discriminate between patients with different postoperative outcomes is most pronounced in the peri-ictal epoch.

The results of machine learning model-building experiments in a large sample (n=294 surgical patients) demonstrate that by using 5 minutes of peri-ictal scalp EEG data, it is possible to build a predictive framework of postoperative seizure outcome with accuracies significantly higher than those obtained from earlier approaches (>90%) and within a range that is likely to be translatable into a clinically useful tool, all while using a data modality that is already part of standard presurgical evaluation globally.

With continued reference to FIG. 1, an embodiment of the method 100 includes development of AI-based epilepsy surgery outcome prediction models using peri-ictal scalp EEG data. In this regard, an exemplary study sample formed of young to middle-aged individuals with equal proportion of males and females, is indicated in Table 1, below. Most patients in the group of patients, including the patient 104, had an abnormal preoperative brain MRI, consistent with the majority of drug resistant temporal lobe epilepsy cases that undergo surgery at epilepsy centers. Inferential testing did not reveal baseline differences between patients in the group of patients who went on to become ‘surgical success’ or ‘surgical failure’ cases. Nonetheless, surgical failure patients tended to be slightly younger with a higher tendency to have a normal preoperative brain MRI and non-localizable seizures on preoperative scalp EEG.

TABLE 1
Patient Characteristics. Adjusted p values represent the result of a
Bonferroni correction for 14 individual comparisons. For continuous
variables, means are provided with standard deviations within brackets.

| | | | Surgical | Surgical | Adjusted |
|---|---|---|---|---|---|
| Variable | | Combined | Success | Failure | p value |
| N | | 294 | 170 | 124 | |
| Age at surgery (Years) | | 37.3 (15.3) | 39.5 (15.5) | 34.3 (14.7) | 0.06 |
| Female sex (%) | | 50 | 49.4 | 50.8 | 1 |
| Abnormal MRI (%) | | 77.9 | 81.2 | 73.4 | 1 |
| Duration of Epilepsy (Years) | | 17.5 (13.5) | 18.5 (14.5) | 16 (11.8) | 1 |
| Monthly Seizure Frequency | | 15.4 (46.3) | 15.2 (53.7) | 15.8 (33.6) | 1 |
| Has had a GTC before surgery (%) | | 81.3 | 77.6 | 87.1 | 0.4 |
| Left sided surgery (%) | | 55.1 | 54.1 | 56.5 | 1 |
| Length of randomly selected seizure | | 1.56 (1.43) | 1.5 (1.48) | 1.64 (1.34) | 1 |
| from preop EEG (minutes) | | | | | |
| Follow-up duration when outcome | | 3.42 (1.96) | 3.42 (1.82) | 3.42 (2.14) | 1 |
| status defined (Years) | | | | | |
| Causes of | MCD^{†} | 64 | 61.8 | 66.9 | 1 |
| seizures | MTS^{†} | 28.2 | 29.4 | 26.6 | 1 |
| (%) | Cryptogenic | 7.5 | 7.6 | 7.3 | 1 |
| Presence of non-localizable seizures on | | 19.4 | 14.1 | 26.2 | 0.1 |
| preoperative EEG (%) | | | | | |
| Interictal | Bilateral epileptiform | 18.7 | 14.7 | 24.2 | 0.6 |
| discharges | discharges | | | | |
| (%) | Ipsilateral (at least 80%) | 72.8 | 78.2 | 65.3 | |
| | None | 8.5 | 7.1 | 10.5 | |

^{†}indicates that MCD and MTS were known etiologies of seizures either alone or in combination.

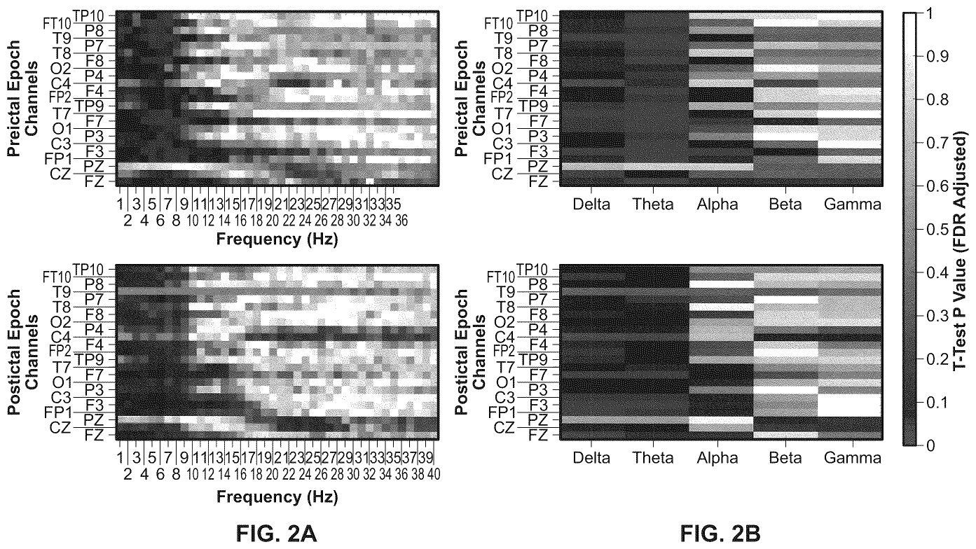

In an initial screening test, statistically significant differences between surgical success and surgical failure cases were investigated. In this regard, heatmaps were plotted based on electrode-frequency pairs as shown in FIG. 2A, and based on electrode-frequency band pairs as shown in FIG. 2B. FIGS. 2A and 2B indicate a screening approach to detect differences in the power spectra between surgical Failure (n=124) and surgical success (n=170) groups. In FIG. 2A, every box in the heatmap represents a t-test of the spectral power for each electrode-frequency pair. In FIG. 2B, every box in this heatmap represents a t-test of the spectral power for each electrode-frequency band pair, the color of the box is based on p-value. Top panels show results from pre-ictal data, bottom panels show results from post-ictal data. As shown in FIGS. 2A and 2B, in the pre-ictal epoch, differences between the two outcome groups were most pronounced in the theta band and were distributed across electrode channels. In the post-ictal epoch, statistically significant differences were less apparent overall but nonetheless were discernable primarily in the delta and theta bands.

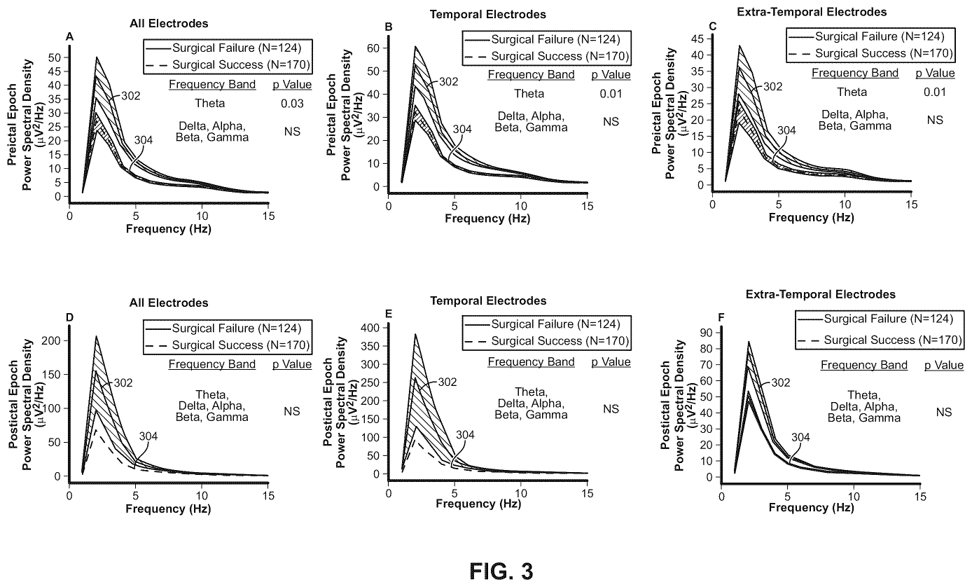

As shown in FIG. 3, power spectral density (μV²/Hz) was plotted across frequencies (Hz) for both surgical success and surgical failure cases to ascertain visually apparent differences in between the two outcome groups on the basis of power spectra. In this regard, when average power spectra for all electrode channels of the EEG data were considered together, the surgical failure cases tended to have higher power in the lower frequency bands, such as delta and theta frequency bands, as compared to surgical success cases, though this was only statistically significant in the theta band in the pre-ictal epoch. When the electrode channels were classified into temporal and extra-temporal electrodes, the same trend was recapitulated.

Surgical failure (line 302, n=124) and surgical success (line 304, n=174) are plotted separately in FIG. 3, groups A-F. FIG. 3 groups A-C show results from pre-ictal EEG data while FIG. 3 groups D-F show results from post-ictal EEG data. FIG. 3 groups A and D show average power spectral densities from all electrodes combined, FIG. 3 groups B and E show averages from all temporal electrodes combined, FIG. 3 groups C and F show averages from all extra-temporal electrodes combined. Shaded areas represent standard error of the mean. Inset tables show p values (adjusted for multiple comparisons for each table) of band-wise power spectral density for the averaged channels.

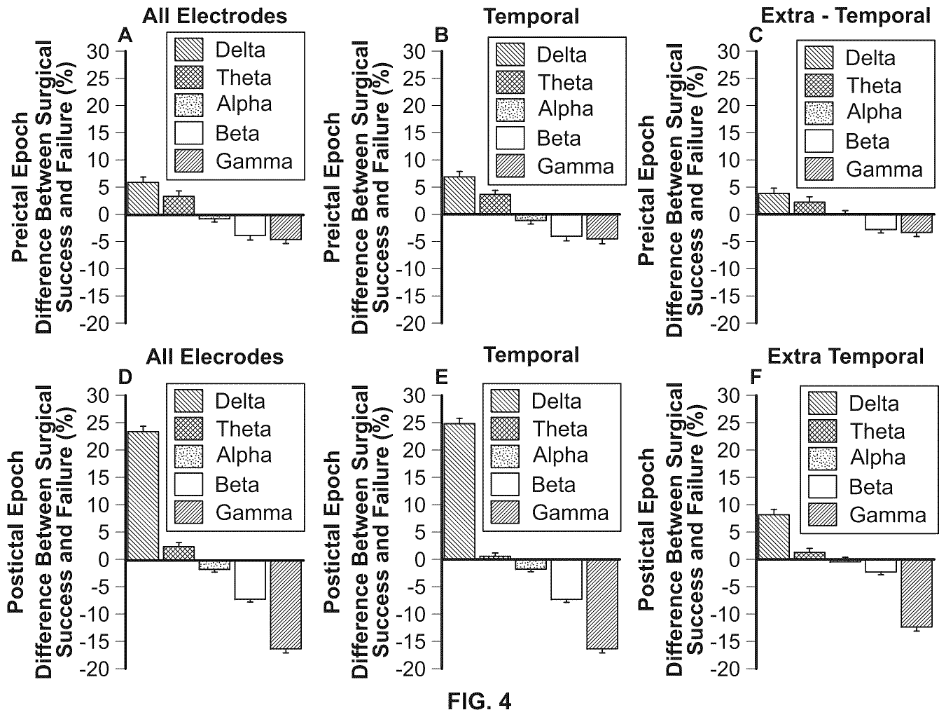

FIG. 3 shows the differences in the absolute power spectral density for different outcome groups across frequencies. Ascertaining the proportional representation of a given frequency band included determining an area under the curve of the power spectral density versus frequency plot, and calculating the proportional area for each frequency band. As shown in FIG. 4, differences in the proportional representation of each frequency band between surgical success and surgical failure groups were determined in groups A-F, including for different electrode groups.

In this regard, the area under the curve of the power spectral density versus frequency plots depicted in FIG. 3 was calculated, and a proportion of the area for each frequency band was determined. More specifically, with reference to FIG. 3, the value on the y-axis is calculated as [proportional representation of the frequency band in the surgical failure group]-[proportional representation of the frequency band in the surgical success group]. Error bars represent 95% confidence intervals for the difference between two proportions. Groups A-C in FIG. 3 represent results from the pre-ictal epoch, and groups D-F in FIG. 3 represent results from the post-ictal epoch.

With reference to FIG. 4, the surgical failure group had markedly higher proportional representation of the delta band in both pre-ictal and post-ictal epochs though the trend was more pronounced in the post-ictal epoch. At the same time, the proportional representation of the gamma band was markedly lower in the surgical failure group than the surgical success group. Once again, this trend was seen in both the pre-ictal and post-ictal setting but was more pronounced post-ictally.

Multiple binary classifiers were built to predict postoperative seizure outcome using scalp EEG derived power spectral density features, as shown in the top three rows of Table 2, below. Multiple classifier types may be fit to this data with excellent meaningful accuracy measures. The best performing model is a version of a Light Gradient Booster Machine (LGBM). As a model built exclusively using peri-ictal scalp EEG features, the LGBM classifier had a mean accuracy (based on 4-fold cross-validation) of 94.5% and out-of-group testing set prediction accuracy of 91.9%. The winning model was based on 34 EEG features which were ranked in importance based on a permutation-based approach, indicated in Table 2.

TABLE 2
Feature importance of the EEG-derived features of the winning
model to predict postoperative seizure outcome. Total number
of features for this model is 34. Feature importance is calculated
using a permutation method. Values of each feature in the training
set are randomly shuffled and the resulting decrement in model
accuracy is logged and then averaged over 10 shuffles. Features
for which shuffling causes a larger decrement in accuracy are
considered more important. Note that the importance of any
one feature is given relative to the most important feature
(i.e. right-most row does not sum to 1).

| Electrode- | Pre-ictal/ | Feature | Relative |
|---|---|---|---|
| Frequency Pair | Post-ictal | Priority | Importance |
| F7 - 27 Hz | Post-ictal | 1 | 100 |
| FT10 - 25 Hz | Pre-ictal | 2 | 77 |
| C4 - 3 Hz | Pre-ictal | 3 | 72 |
| T8 - 15 Hz | Pre-ictal | 4 | 61 |
| Fz - 4 Hz | Pre-ictal | 5 | 51 |
| F3 - 28 Hz | Pre-ictal | 6 | 49 |
| F4 - 34 Hz | Pre-ictal | 7 | 44 |
| P7 - 23 Hz | Post-ictal | 8 | 42 |
| C3 - 6 Hz | Pre-ictal | 9 | 40 |
| Pz - 27 Hz | Post-ictal | 10 | 37 |
| Cz - 21 Hz | Pre-ictal | 11 | 35 |
| Fz - 30 Hz | Post-ictal | 12 | 35 |
| P8 - 25 Hz | Pre-ictal | 13 | 33 |
| Pz - 34 Hz | Pre-ictal | 14 | 32 |
| P8 - 2 Hz | Pre-ictal | 15 | 32 |
| P4 - 2 Hz | Pre-ictal | 16 | 30 |
| T9 - 3 Hz | Pre-ictal | 17 | 25 |
| P8 - 5 Hz | Post-ictal | 18 | 23 |
| Cz - 32 Hz | Post-ictal | 19 | 19 |
| P7 - 32 Hz | Pre-ictal | 20 | 19 |
| P7 - 10 Hz | Post-ictal | 21 | 18 |
| P7 - 29 Hz | Pre-ictal | 22 | 16 |
| P8 - 36 Hz | Pre-ictal | 23 | 16 |
| P7 - 7 Hz | Pre-ictal | 24 | 11 |
| O1 - 25 Hz | Pre-ictal | 25 | 9 |
| FT10 - 5 Hz | Pre-ictal | 26 | 9 |
| P4 - 25 Hz | Post-ictal | 27 | 7 |
| FT10 -1 Hz | Pre-ictal | 28 | 4 |
| FT10 - 24 Hz | Post-ictal | 29 | 4 |
| T9 - 31 Hz | Pre-ictal | 30 | 2 |
| T9 - 33 Hz | Post-ictal | 31 | 2 |
| T9 - 24 Hz | Pre-ictal | 32 | 0 |
| F8 - 20 Hz | Pre-ictal | 33 | 0 |
| P7 - 17 Hz | Pre-ictal | 34 | 0 |

With reference to Table 3, below, in the winning model, 70% of features were from the pre-ictal period and 30% were from post-ictal period. 62% of features were from temporal electrodes while 38% were from extra-temporal electrodes. 56% of features were from the beta band, while 12%, 12%, 5%, and 3% were from gamma, theta, delta, and alpha bands respectively.

TABLE 3
Summary of different binary classifier models to predict postoperative seizure outcomes
after temporal lobe resection based on peri-ictal scalp EEG derived EEG features and/or
clinical variables. Accuracy, precision, recall, and AUC of ROC are based on testing
of the model on an outcome-stratified hold-out testing set (25% of total data).

| Model Features | Candidate Model | Accuracy | Precision | Recall | AUC of ROC |
|---|---|---|---|---|---|
| Peri-ictal scalp EEG | LGBM, 34 EEG | 91.9 | 95.1 | 90.7 | 0.977 |
| features only | features | | | | |
| Peri-ictal scalp EEG | RFC (with PCA, 57 | 81.1 | 79.6 | 90.7 | 0.901 |
| features only | features) | | | | |
| Peri-ictal scalp EEG | CatBoost, 28 EEG | 90.5 | 89.1 | 95.3 | 0.988 |
| features only | features | | | | |
| Peri-ictal scalp EEG | RFC (with PCA for | 79.7 | 79.2 | 88.4 | 0.873 |
| features AND clinical | EEG features, 12 | | | | |
| variables from | clinical features) | | | | |
| prevailing nomogram | | | | | |
| Clinical variables from | Logistic Regression | 62.1 | 67.4 | 67.4 | 0.642 |
| prevailing nomogram | | | | | |

LGBM: Light Gradient Booster Machine. RFC: Random Forest Classifier. CatBoost: Category Boost.

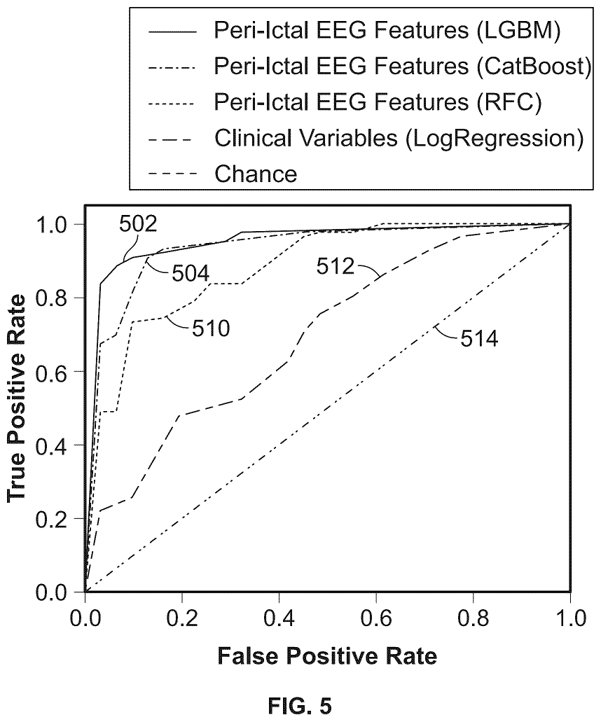

As shown in FIG. 5, a Receiver Operating Characteristic (ROC) curve was plotted to quantify the discriminatory ability of the winning EEG-augmented model over a range of discrimination thresholds. The ROC curve analysis indicated in FIG. 5 shows that both the clinical variables model and the EEG-augmented model perform better than chance, and the EEG-augmented model is the most discriminatory across the range of discriminatory thresholds.

In this manner, FIG. 5 depicts performance characteristics of EEG-augmented and non-augmented surgical outcome prediction models, including ROC curves showing performance of the EEG-augmented models, where curve 502 indicates a Light Gradient Boost Machine model, curve 504 indicates a CatBoost model, curve 510 indicates a Random Forest Classifier model, curve 512 indicates a comparison model built on clinical variables alone, and curve 514 indicates a policy based on chance.

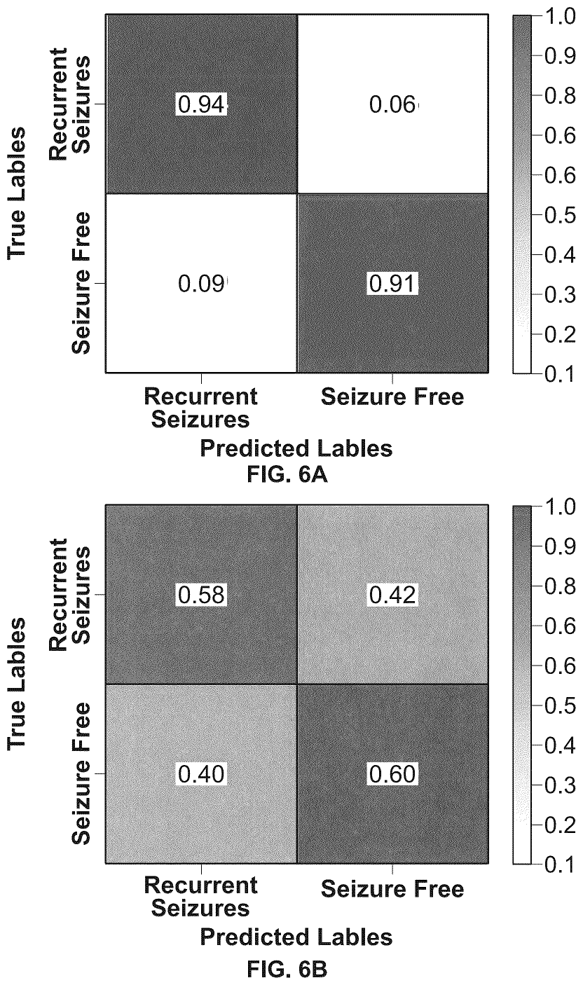

As shown in FIGS. 6A and 6B, normalized confusion matrices were also generated to quantify the likelihood that erroneous predictions would be false positives (i.e. the model erroneously predicts seizure freedom) or false negatives (i.e. the model erroneously predicts seizure recurrence). On the hold-out testing set, the EEG-augmented model was slightly more likely to erroneously predict seizure freedom (i.e. the model slightly overestimated the therapeutic effect of surgery). The matrix depicted in FIG. 6A corresponds to the LGBM model, and proportions in horizontal dimension sum to 1. FIG. 6B depicts a normalized confusion matrix for the non-EEG-augmented outcome prediction model built on descriptive clinical features alone, and proportions in horizontal dimension sum to 1.

Decision Curve Analysis (DCA) was performed to quantify the clinical usefulness of the EEG-augmented outcome prediction approach in terms of net benefit across a range of probability thresholds, as shown in FIG. 5. Notably, the probability threshold range of 30-70% is particularly interesting as this is the range in which an outcome prediction model is useful. In this regard, clinicians would be hesitant to change treatment plans based on automated outcome prediction approaches outside of this threshold range. For example, if the clinician is starting with an expectation, based on their clinical judgment, that the patient has less than a 30% chance of becoming seizure-free with surgery, it is unlikely that a model prediction will sway them into deciding to resect. If the baseline estimate by the clinician is greater than 70%, they are likely to just proceed without needing a model to inform them further. Over this range of probability thresholds, the use of the EEG-augmented approach consistently increased net benefit compared to use of the clinical variables nomogram or a ‘treat all’ strategy indicated in FIG. 5.

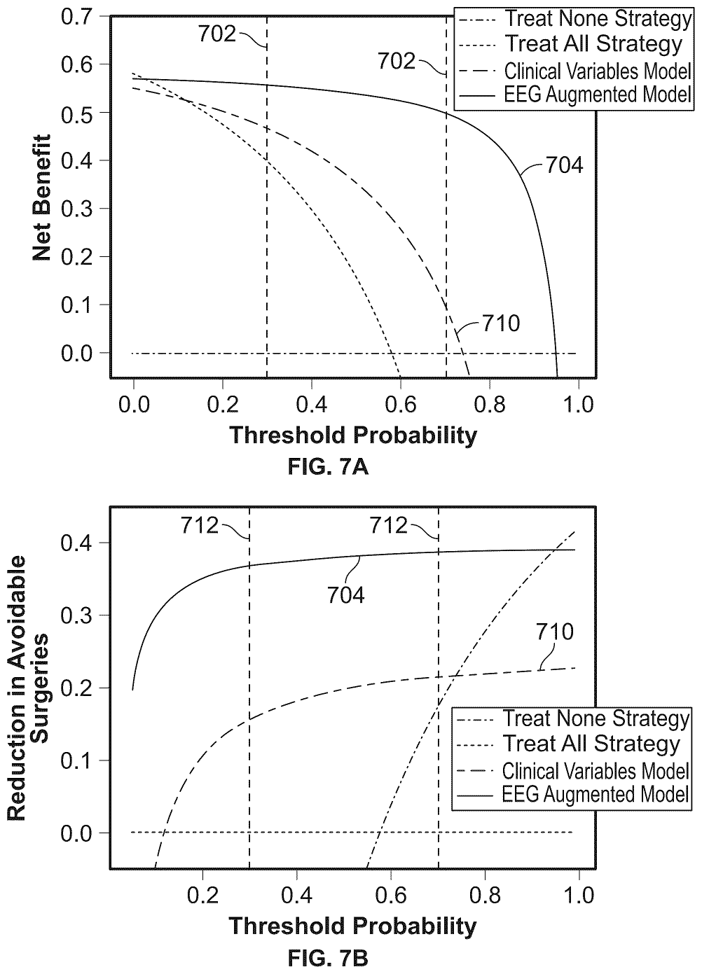

The use of the EEG-augmented approach also maximized the number of unnecessary surgeries that could be avoided, as indicated in FIG. 7A. Across the relevant range of probability thresholds, the EEG-augmented approach would decrease the number of unnecessary surgeries by approximately 40% compared to a ‘treat all’ strategy and approximately 20% compared to an approach based on the clinical variables nomogram.

FIG. 7A depicts a net benefit plotted against a range of threshold probabilities. In this regard, net benefit at a given threshold probability (pt) quantifies the ‘benefits’ (true positives) after accounting for the harms of false positives (weighted by the threshold probability), and may be calculated using the following equation (1):

$$NB ( p t ) = TP - ( FP \times W ) ) / N$$

With reference to equation (1) above, TP=true positive, FP=false positive. W=“weighting factor”=(pt)/(1−(pt)), N=total number of patients. Threshold probability is the probability where a clinical decision-maker would be indifferent when choosing between two actions (i.e. to resect or not). Within the range of threshold probabilities in which the model is most likely to be utilized (30-70%, dashed vertical lines 702 in FIG. 7A), the EEG-augmented model clearly outperforms the non-EEG-augmented model, as a curve 704 generated by the EEG-augmented model is consistently higher than a curve 710 generated by the non-EEG-augmented model.

FIG. 7B depicts a net reduction in unnecessary surgeries plotted against threshold probability for both outcome prediction models. Net reduction in avoidable surgeries for a prediction model (i.e. surgeries that the model would have correctly predicted to be unsuccessful) is calculated as (net benefit of using prediction model—net benefit of treat all strategy)/(pt)/(1−(pt)). Within the range of threshold probabilities in which the model is most likely to be utilized (30-70%, dashed vertical lines 712), the EEG-augmented model clearly outperforms the non-EEG-augmented model, as a curve 714 generated by the EEG-augmented model is consistently higher than a curve 720 generated by the non-EEG-augmented model.

The peri-ictal window is particularly valuable for electrophysiological studies in DRE. In this regard, for example, in the window of time immediately before a seizure occurs (“before rapid discharge” or “BRD”), there is an observable synchronization event between mesial temporal structures. As such, the periods of time immediately before and after a seizure represent a brain state that is both quantitatively and qualitatively distinct from the brain state hours before or after a seizure (the latter more commonly referred to as the “interictal” period.) Complementarily, preoperative functional connectivity (based on implanted EEG electrodes) can be used to discriminate between patients who will go on to be surgical success or surgical failure cases.

The value of peri-ictal EEG data becomes particularly clear when compared with attempts to use inter-ictal EEG to predict postoperative seizure outcomes in DRE patients. For example, when using visually reviewed ‘normal’ resting state pre-operative EEG (i.e. inter-ictal EEG taken hours before/after a seizure), tested ML classifiers were able to predict postoperative seizure outcome with modest accuracy (AUC 0.78). The use of peri-ictal EEG substantially changed the predictive ability of the models (AUCs >0.95 for multiple models). This comparison suggests that future electrophysiological investigations in DRE, particularly in terms of understanding differential responses to surgical therapies, should focus on the peri-ictal window.

Machine learning classifiers are able to discern differences in power spectral features that are not apparent on visual inspection with quantifiable accuracy. In this regard, direct visual comparison of the power spectral density plots of surgical success and surgical failure patients revealed minimal differences between the two groups depicted in FIG. 3. However, multiple different ML classifiers were able to use power spectral features to accurately distinguish these patient groups.

Notably, physician interpretation of scalp EEG is a valuable portion of presurgical evaluations for the purpose of seizure localization, but not postsurgical seizure outcome prediction. Review of the peri-ictal scalp EEG features reveals that there are several dozen features that help discriminate between the two outcome groups with complex interdependencies, for example, increased delta power in the temporal electrodes, increased theta power across the scalp, decreased proportional representation of the gamma band. A physician seeking to make a prediction of postsurgical outcome on the basis of a single preoperative scalp EEG would have to consider all these features simultaneously in order to make a prediction for any one patient. As such, a human interpreter would not be reasonably able to make an accurate prediction given the underlying complexity.

Also, the machine learning classifier offers a consistent and quantifiable accuracy for each prediction that is made. The predictive accuracy of electrophysiologists interpreting clinical EEG data is difficult to quantify, highly variable, and likely to change as a function of experience and human factors (e.g. fatigue, bias). More generally, different expert epileptologists reviewing the same patient case are likely to give highly variable estimates of postoperative seizure outcome with poor correlation with real outcomes.

The method 100 offers machine-learning enabled postoperative seizure control prediction in the context of DRE that is both highly accurate and makes use of data captured noninvasively and inexpensively during routine presurgical evaluation. In this regard, the method 100 includes the use of peri-ictal scalp EEG derived features in a machine-learning enabled predictive framework to achieve clinical translation into a useful presurgical tool, and reduce avoidable surgeries.

DCA may be utilized to evaluate the likely clinical usefulness of the method 100. In this regard, DCA provides a meaningful way of quantifying and comparing the clinical usefulness of prediction models, including within the context of neurosurgery. Applied to the method 100, DCA shows increased net benefit (i.e. true positives) when using an EEG-augmented model which translates into a 20-40% reduction in avoidable surgeries compared to decision-making strategies without the EEG-augmented model.

An automated machine-learning enabled artifact annotator is employed in preprocessing the raw EEG data from an EEG apparatus 124. The use of automation in this context not only allows for the rapid preprocessing of a large amount of data. In the present example, approximately 1500 minutes of raw EEG data would otherwise have had to undergo manual human inspection. The artifact annotator also ensures that a consistent standard for artifact status is applied to all data in the study, thereby increasing reproducibility. These automated methods also support improved scientific rigor through the application of two independent and complementary validation strategies: cross-validation and out-of-group testing. The latter is rarely implemented in prior studies due to the requirement for sufficiently large datasets to permit reliable data splitting. By leveraging this scalable, automated pipeline, the method 100 enables relatively large, comprehensive analysis of EEG data from epilepsy patients who have undergone brain resection.

In an example study at block 110 of the method 100, all EEG studies were captured at a single institution as part of an institutional protocol for presurgical evaluation. In an alternative embodiment, the method 100 includes incorporating EEG data from a plurality of different institutions. Notably, while the EEG apparatus 124 is a scalp EEG recording system configured for analyzing temporal lobe epilepsy cases, which represent the most frequently encountered surgical epilepsy in most academic centers, the method 100 may additionally or alternatively be expanded to extra-temporal epilepsies using a different configuration of the EEG apparatus 124.

Furthermore, while the method 100 as described includes optimizing the prediction of seizure outcome after resective surgery, brain resection is not the only surgical option available for DRE, and the method 100 may additionally or alternatively direct predictive action toward non-resective (and thus less invasive) surgical options such as, for example and without limitation, vagal nerve stimulation, responsive neuromodulation, anterior nucleus of the thalamus deep brain stimulation (ANT-DBS), and laser interstitial ablation without departing from the present disclosure. In a further embodiment where the model is trained to perform various or comparative predictions, the method 100 may include directing patients toward non-resective treatments that are likely to provide comparable or greater benefit without the risk profile of resective surgery. In this regard, the method 100 may include performing comparative predictions using the model to determine which of the multiple surgical treatments is most likely to benefit a specific patient based on preoperative EEG and clinical features. As such, the method 100 provides a predictive framework that uses a combination of clinical variables and peri-ictal scalp EEG data for high accuracy postoperative predictions. The method 100 is likely to be highly clinically translatable as scalp EEG is often acquired for surgical candidates and is non-invasive and inexpensive.

In the described experimentation, as an exemplary embodiment of the method 100 depicted in FIG. 1, retrospective data were captured from patients who had undergone temporal lobe resection for drug resistant temporal lobe epilepsy. To be included in the study, patients had to have undergone a preoperative scalp EEG evaluation during with a recorded seizure during that time. Post-surgical seizure outcome at the last follow-up was used as the basis of outcome classification. Patients who were completely seizure free postoperatively (equivalent to Engel Classification Class I or International League Against Epilepsy Classes 1 and 2) were considered ‘surgical success’ cases while those with persistent seizures of any severity (Engel II-IV or ILAE 3-6) were considered ‘surgical failure’ cases. To ascertain differences in the baseline characteristics of patients in the surgical success and surgical failure groups, inferential tests were applied; two-sided t tests were applied for comparison of means, Fisher Exact tests were applied for comparison of proportions when only two categories were present, Chi square tests were applied when more than 2 categories were present. The study was conducted under an Institutional Review Board approved outcomes registry protocol.

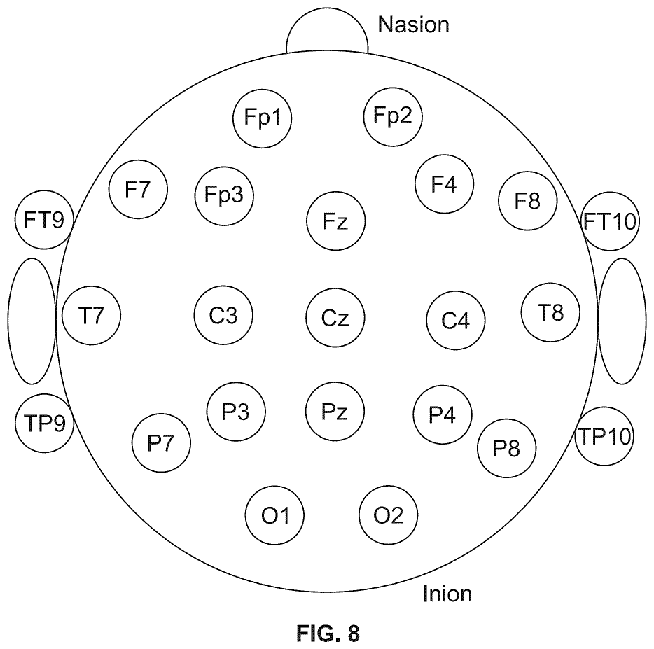

As an exemplary embodiment of the method 100, EEG studies were recorded using an extended 10-20 electrode placement scheme at a sampling rate of 200 Hz. As shown in FIG. 8, the electrode placement included 23 locations: Fz, Cz, Pz, Fp1, Fp3, C3, P3, 01, F7, T7, TP9, FT9, P7, Fp2, F4, C4, P4, O2, F8, T8, P8, FT10, TP10. For certain analyses, ‘temporal’ and ‘extra-temporal’ electrodes were separately considered. For those analyses, the ‘temporal’ electrodes were TP10, FT10, P8, T9, P7, T8, F8, TP9, T7, F7. The reference electrodes for the headboxes were C3 and C4. No re-referencing was performed.

Referring back to FIG. 1, in embodiments of the method 100 where patients have multiple seizures over several days of observation, a standardized strategy may be employed to determine which seizure should be selected for each patient. In this regard, seizures captured later in the course of a patient's inpatient monitoring stay may have more or less value to an outcome prediction model compared to those captured earlier in the stay. Additionally, some patients have multiple seizure types as classified by scalp EEG. In a prior analysis of 386 temporal lobe epilepsy patients, while the vast majority (72%) had a single type of ictal EEG pattern on preoperative evaluation, a minority had multiple ictal patterns (the presence of multiple ictal patterns was not an independent predictor of postoperative surgical outcome.) In embodiments, the method 100 employs a randomization strategy in order to address these sources of data variability at the individual patient level. As such, for each patient across the cohort, a single seizure at random and captured the EEG file in European Data Format (EDF) for subsequent analysis may be selected.

In the embodiment of the method 100, EEG files were annotated with ‘on’ and ‘off’ labels around the time of a seizure as part of the clinical workflow of patients undergoing epilepsy surgery evaluation. The ‘on’ label marks the time of seizure onset (i.e. time at which a trained EEG technologist can see the beginning of an organizing seizure activity on an EEG) while the ‘off’ label marks the post-ictal time point where no more organized seizure activity is ascertainable. Regarding technician-annotated data used for training the model at block 122, large prospective studies have reported interrater agreement rates greater than 95% between trained EEG technologists and clinical neurophysiologists in the detection of seizures among epilepsy patients. For each selected seizure, 2 minutes of EEG data immediately preceding the annotated seizure onset (“on” label) were extracted as the pre-ictal period, and 3 minutes of EEG data immediately following the annotated seizure termination (“off” label) were extracted as the post-ictal period.

In the embodiment of the method 100, preprocessing of EEG data may include removal of additional electrode channels, harmonization of electrode labels, automated artifact annotation, generation of power spectra, and/or feature extraction. Artifact detection and annotation can include using an automated pipeline including a trained and validated support vector machine (SVM) classifier which samples the raw EEG time series data and annotates each second as either artifact free or artifactual. Subsequent analyses may then be performed exclusively on artifact-free data. When applied to the pre-ictal data (2 minutes of raw data per patient), the artifact detector identified on average 77 seconds of artifact free data per patient in the surgical success group (95% CI 73-80) and 74 seconds in the surgical failure group (95% CI 71-79). When applied to the post-ictal data (3 minutes of raw data per patient), the artifact detector identified on average 88 seconds of artifact free data per patient for the surgical success group (95% CI 81-94) and 82 seconds for the surgical failure group (95% CI 76-87).

In the embodiment of the method 100, artifact free EEG data retained after annotation by the SVM classifier was utilized to generate power spectral density information for each patient from both the pre-ictal and post-ictal epochs. Specifically, a periodogram was gathered from artifact-free segments for each channel and these were then averaged to produce the power spectral density across all channels in each frequency bin for a given patient using the first 40 frequency bins (1-40 Hz) for model building. This resulted in a [23 channel×40 frequency bin] matrix containing pre-ictal data, and another [23 channel×40 frequency bin] matrix containing post-ictal data. A Z-score normalization was applied on a per electrode and per frequency basis for machine learning applications.

The embodiment of the method 100 included nine clinical features: preoperative monthly seizure frequency, occurrence of generalized convulsion at any time before surgery (yes/no), cause of seizures (mesial temporal sclerosis/malformation of cortical development/stroke/tumor/other), years of epilepsy duration at time of surgery, gender (male/female), MRI findings (normal/abnormal), EEG seizure localization (always localizable/sometimes not localizable), and interictal epileptiform discharges (>80% unilateral/bilateral/no interictal epileptiform discharges). In addition, the clinical features employed in the embodiment of the method 100 included side of surgery (left/right), age at time of surgery in years, and follow-up period (at which seizure outcome was assigned) in years. In total, 12 clinical features were included.

The embodiment of the method 100 includes flattening and horizontally concatenating the pre-ictal and post-ictal EEG data, which were in the form of two separate [23×40] arrays into a single one-dimensional array [1×1840]. The 12 clinical variables were then horizontally concatenated to this array to make an “EEG plus clinical variables” array for each patient [1×1852]. The final data matrix for all 294 patients was thus created [294 patients×1852 features].

In the embodiment of the method 100, machine learning at block 122 was conducted using an AutoML workflow. The AutoML workflow offers an automated method that fits multiple candidate classifier models to a dataset and uses a grid-search strategy to select an optimal set of hyperparameters. The AutoML implementation generates a model that can be explored, validated, and optimized by an investigator.

In the embodiment of the method 100, a two-fold approach was implemented to model validation of the machine learning at block 122. First, a stratified k-fold cross-validation (k=4) strategy was implemented. In the context of this cross-validation strategy, accuracy was calculated as the quotient of out-of-fold predicted labels that matched the true label divided by the total number of samples. Area under the receiver operating curve (AUC-ROC), precision, and recall were also calculated. Secondly, an ‘out of group’ testing strategy was implemented where the model using a stratified training set containing 75% of the total dataset, and retained 25% of the dataset was built as an “out of group” testing set.

In the embodiment of the method 100, DCA was implemented as supplanting statistical model parameters (e.g., AUC) with individual preference and procedure outcomes to quantify the clinical usefulness of a model. This DCA quantification was accomplished by calculating a clinical “Net Benefit” (NB) for one or more prediction models in comparison to default strategies of treating all or no patients, or treatment based on other tests, as indicated by the following equation (2):

$$Net Benefit ( p t ) = \frac{TP}{N} - ( \frac{FP}{N} * \frac{p t}{1 - p t} )$$

With reference to equation (2) above, TP=true positives, FP=false positive, N=total number of patients, pt=probability threshold. In the embodiment of the method 100, DCA was implemented as supplanting statistical model parameters.

In effect, the Net Benefit is a single number that incorporates true positive rate while penalizing for the harms of false positives. The probability threshold is the probability at which a clinical decision maker would be indifferent between two possible actions. In the DCA, the Net Benefit provided by a clinical prediction model was plotted over a range of clinically relevant threshold probabilities; curves that lie higher on the plot represent more clinically useful prediction strategies. To plot a DCA, the range of pt should be clinically defined a priori as the range within which a guidance on risk could be helpful: below the minimum pt, a temporal lobe resection is typically not recommended (success chance is too low); above the max pt, temporal lobe resection is usually offered; in-between is the gray area where a model could inform the decision. If a model trained at block 122 has the highest net benefit across the entire pre-defined range of pts, the model used when clinically feasible.

DCA also allows for estimation of the reduction in unnecessary interventions (i.e. likely unsuccessful surgeries that could have been avoided by using a given prediction model), as indicated by the following equation (3):

$$Reduction in likely unsuccessful ( i . e . avoidable ) surgeries = \frac{NB of prediction model - NB of ‘ treat all ’ strategy}{\frac{p t}{1 - p t}}$$

While various features are presented above, it should be understood that the features may be used singly or in any combination thereof. Further, it should be understood that variations and modifications may occur to those skilled in the art to which the claimed examples pertain.

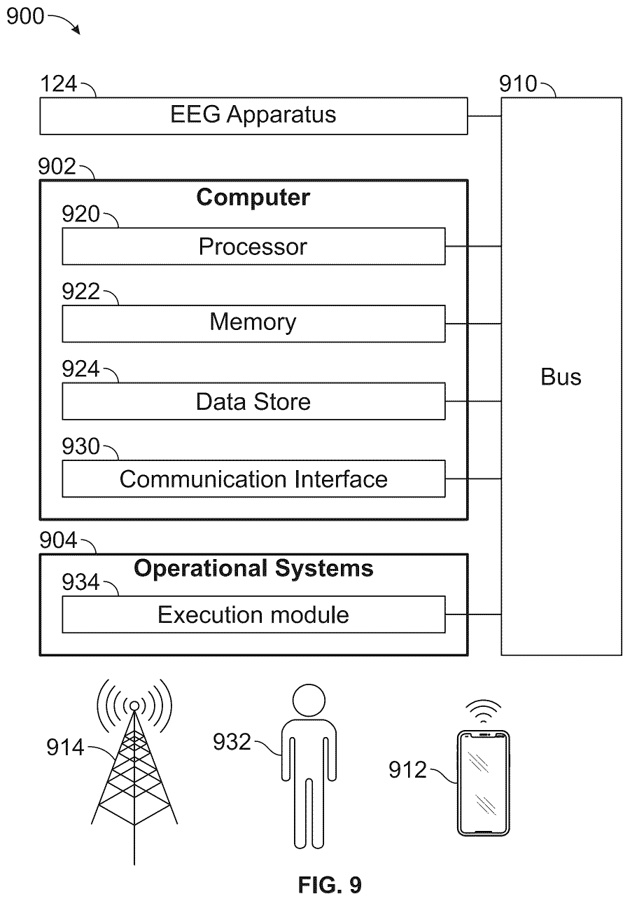

FIG. 9 is an exemplary component diagram of a system 900 for predicting a seizure occurrence or a surgical outcome, according to one aspect. The system 900 includes the EEG apparatus 124, a computer 902, and operational systems 904. The EEG apparatus 124, the computer 902, and the operational systems 904 may be interconnected by a bus 910. The components of the system 900, as well as the components of other systems, hardware architectures, and software architectures discussed herein, may be combined, omitted, or organized into different architectures for various embodiments. The computer 902 may be implemented with a device or remotely stored.

The computer 902 may be configured to execute the method 100, implemented as a part of the EEG apparatus 124, and support elements of the method 100 from conducting EEG studies at block 110, to training models at block 122. The computer 902 may be implemented as part of a telematics unit or an electronic control unit among other potential aspects of the EEG apparatus 124. In other embodiments, the components and functions of the computer 902 can be implemented with other devices such as a portable device 912, database, remote server, or another device connected via a network (e.g., a network 914).

The computer 902 may be capable of providing wired or wireless computer communications utilizing various protocols to send and receive electronic signals internally to and from components of the system 900. Additionally, the computer 902 may be operably connected for internal computer communication via the bus 910 (e.g., a Controller Area Network (CAN) or a Local Interconnect Network (LIN) protocol bus) to facilitate data input and output between the computer 902 and the components of the system 900.

The computer 902 includes a processor 920, a memory 922, a data store 924, and a communication interface 930, which are each operably connected for computer communication via the bus 910 and/or other wired and wireless technologies. The communication interface 930 provides software and hardware to facilitate data input and output between the components of the computer 902 and other components, networks, and data sources, which will be described herein.

The computer 902 is also operably connected for computer communication (e.g., via the bus 910 and/or the communication interface 930) to one or more operational systems 904. The operational systems 904 can include, but are not limited to, any automatic or manual systems that can be used to enhance the EEG apparatus 124, and facilitate operation of the EEG apparatus 124 by a user 932. The operational systems 904 include an execution module 934. The execution module 934 may monitor, analyze, or operate the EEG apparatus 124 to some degree. For example, the execution module 934 may store, calculate, and provide information about the EEG apparatus 124, such as previous usage statistics, including sensor data from previous use.

The operational systems 904 also include and/or are operably connected for computer communication to the EEG apparatus 124. For example, one or more sensors or electrodes of the EEG apparatus 124 may be incorporated with the execution module 934 to monitor characteristics of the EEG apparatus 124, including when conducting an EEG study at block 110, and other aspects of the EEG apparatus 124. In another embodiment, the EEG apparatus 124 may communicate with one or more devices or services (e.g., a wearable computing device, non-wearable computing device, cloud service, etc.) to perform processing or communication functions.

The EEG apparatus 124, the computer device 902, or the operational systems 904 are also operatively connected for computer communication to and via the network 914. The network 914 is, for example, a data network, the Internet, a wide area network (WAN) or a local area (LAN) network. The network 914 serves as a communication medium to various remote devices (e.g., databases, web servers, remote servers, application servers, intermediary servers, client machines, or other portable devices).

As such, the system 900 facilitates improved clinical decision-making in the context of epilepsy surgery by generating and deploying a predictive model for seizure outcomes that incorporates peri-ictal EEG data acquired using a scalp EEG system, such as the EEG system 124, and integrates relevant clinical variables. Still another aspect involves a non-transitory computer-readable medium including processor-executable instructions configured to implement one aspect of the techniques presented herein.

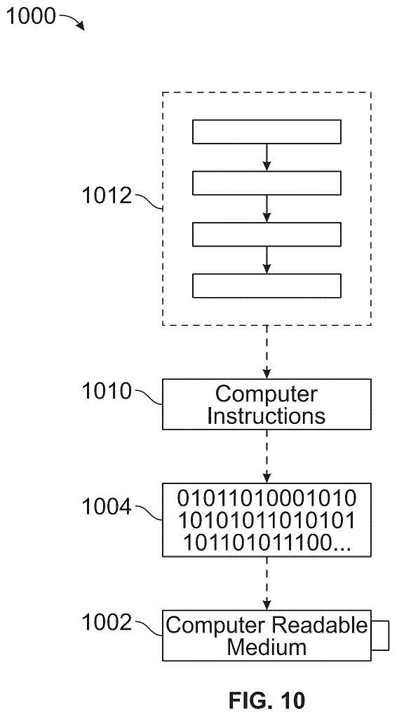

In this regard, an aspect of a computer-readable medium or a computer-readable device devised in these ways is illustrated in FIG. 10, where an implementation 1000 includes a computer-readable medium 1002, such as a CD-R, DVD-R, flash drive, a platter of a hard disk drive, etc., on which is encoded computer-readable data 1004. This encoded computer-readable data 1004, such as binary data including a plurality of zero's and one's as shown in 1004, in turn includes a set of processor-executable computer instructions 1010 configured to operate according to one or more of the principles set forth herein. In this implementation 1000, the processor-executable computer instructions 1010 may be configured to perform a method 1012, such as the method 100 of FIG. 1. In another aspect, the processor-executable computer instructions 1010 may be configured to implement a system, such as the system 900 of FIG. 9. Many such computer-readable media may be devised by those of ordinary skill in the art that are configured to operate in accordance with the techniques presented herein.

While specific embodiments are shown and described herein, it is contemplated that alternative embodiments exist that employ alternative materials, mixtures, proportions, sizes, etc. without departing from the spirit and/or scope of the innovation as described in detail. These alternative embodiments are to be included within the spirit and scope of the innovation as described and claimed herein.

Although the subject matter has been described in language specific to structural features or methodological acts, it is to be understood that the subject matter of the appended claims is not necessarily limited to the specific features or acts described above. Rather, the specific features and acts described above are disclosed as example aspects.

1. A method of predicting a seizure occurrence or a surgical outcome, the method comprising:
monitoring a patient using an electroencephalography (EEG) system, including recording EEG data that indicates a seizure;
extracting a peri-ictal segment of the EEG data that includes a pre-ictal period immediately preceding the seizure, or a post-ictal period immediately following the seizure;
processing the peri-ictal segment, including generating an electrode-wise power spectral density (PSD) feature across a frequency band defined by a delta frequency range, a theta frequency range, an alpha frequency range, a beta frequency range, or a gamma frequency range; and
inputting the PSD feature into a machine learning system, wherein the machine learning system is trained to predict a seizure occurrence or a surgical outcome of the patient based on the PSD feature.

2. The method of claim 1, wherein processing the peri-ictal segment further comprises:
identifying and removing EEG data that includes an artifact, retaining artifact-free peri-ictal EEG data; and
converting the artifact-free peri-ictal EEG data from a time domain to a frequency domain to generate the PSD feature.

3. The method of claim 1, wherein processing the peri-ictal segment further comprises integrating the PSD feature with a clinical variable associated with the patient, the clinical variable indicating a seizure frequency, history of generalized convulsions, cause of seizures, duration of epilepsy, gender, magnetic resonance imaging data or findings, EEG seizure localization, inter-ictal epileptiform discharge, a side of a surgery, an age at which the surgery was performed, or a follow-up period to the surgery.

4. The method of claim 1, wherein the peri-ictal segment of the EEG data includes both the pre-ictal period and the post-ictal period, the pre-ictal period has a duration of at least 2 minutes immediately preceding the seizure, and the post-ictal period has a duration of at least 3 minutes immediately following the seizure.

5. The method of claim 1, wherein processing the peri-ictal segment comprises generating the PSD feature from post-ictal period EEG data across the delta frequency range, pre-ictal period EEG data across the theta frequency range, or post-ictal period EEG data across the gamma frequency range.

6. The method of claim 1, wherein the machine learning system comprises a Light Gradient Boosting Machine (LGBM) classifier trained on peri-ictal scalp EEG features, wherein the LGBM predicts postoperative seizure outcomes based on the input PSD feature, and performs binary classification that distinguishes between patients likely to achieve seizure freedom and those likely to experience seizure recurrence following resection surgery.

7. The method of claim 1, further comprising:
identifying a group of patients, including the patient, diagnosed with drug resistant epilepsy;
conducting an inpatient video-EEG study for each patient in the group of patients, wherein at least one patient is recorded having multiple seizures;
selecting one of the multiple seizures at random for each patient in the group of patients having multiple seizures recorded;
extracting peri-ictal EEG data corresponding to a period immediately before and after each selected seizure; and
training the machine learning system on the extracted peri-ictal EEG data to predict a postoperative seizure outcome.

8. The method of claim 1, wherein processing the peri-ictal segment comprises identifying temporal electrodes and extra-temporal electrodes that generated the EEG data, calculating an average PSD of the temporal electrodes, calculating an average PSD of the extra-temporal electrodes, and comparing the PSD of the temporal electrodes and the PSD of the extra-temporal electrodes, wherein the comparison of the temporal electrodes and the extra-temporal electrodes forms the PSD feature.

9. The method of claim 1, further comprising:
determining a curve from the peri-ictal segment of the EEG data, wherein the curve indicates PSD versus frequency;
determining an area under the curve within at least one frequency band; and
determining a proportional area for each of the at least one frequency band by dividing the area under the curve within the frequency band by a total area under the curve across all frequency bands.

10. The method of claim 9, wherein the at least one frequency band includes a plurality of frequency bands respectively defined by each of the delta frequency range, the theta frequency range, the alpha frequency range, the beta frequency range, and the gamma frequency range.

11. The method of claim 1, further comprising:
performing a resection surgery on the patient based on the prediction;
conducting longitudinal follow up with the patient, including determining postoperative seizure outcomes based on whether the patient has persistent seizures of any severity; and
training the machine learning system based on the determined postoperative seizure outcome.

12. A system for predicting a seizure occurrence or a surgical outcome, the system comprising:
an electroencephalography (EEG) system that monitors a patient and records EEG data indicative of a seizure;
at least one processor operatively coupled to the EEG system, wherein the at least one processor is configured to:
extract a peri-ictal segment of the EEG data that includes a pre-ictal period immediately preceding the seizure or a post-ictal period immediately following the seizure;
generate a feature representative of the EEG data based on the peri-ictal segment;
integrate the feature with at least one clinical variable associated with the patient, the clinical variable indicating seizure frequency, history of generalized convulsions, cause of seizures, duration of epilepsy, gender, magnetic resonance imaging data or findings, EEG seizure localization, inter-ictal epileptiform discharge, a side of a surgery, an age at which the surgery was performed, or a follow-up period to the surgery; and
input the integrated feature and at least one clinical variable into a machine learning system trained to predict a seizure occurrence or a surgical outcome of the patient.

13. The system of claim 12, wherein the at least one processor is configured to generate an electrode-wise power spectral density (PSD) feature across a frequency band defined by a delta frequency range, a theta frequency range, an alpha frequency range, a beta frequency range, or a gamma frequency range as the feature representative of the EEG data.

14. The system of claim 13, wherein the at least one processor is configured to generate the PSD feature from post-ictal period EEG data across the delta frequency range, pre-ictal period EEG data across the theta frequency range, or post-ictal period EEG data across the gamma frequency range.

15. The system of claim 13, wherein the at least one processor is configured to:
identify and remove EEG data from the peri-ictal segment where the EEG data includes an artifact, and retain artifact-free peri-ictal EEG data; and
generate the PSD feature by converting the artifact-free peri-ictal EEG data from a time domain to a frequency domain.

16. The system of claim 12, wherein the peri-ictal segment of the EEG data includes both the pre-ictal period and the post-ictal period, the pre-ictal period has a duration of at least 1 minute immediately preceding the seizure, and the post-ictal period has a duration of at least 1 minute immediately following the seizure.

17. The system of claim 12, wherein the machine learning system comprises a Light Gradient Boosting Machine (LGBM) classifier trained entirely on peri-ictal scalp EEG features, wherein the LGBM is trained to predict postoperative seizure outcomes based on the input feature, and performs binary classification that distinguishes between patients likely to achieve seizure freedom and those likely to experience seizure recurrence following resection surgery.

18. The system of claim 12, wherein the at least one processor is configured to identify temporal electrodes and extra-temporal electrodes that generated the EEG data, calculate an average PSD of the temporal electrodes, calculate an average PSD of the extra-temporal electrodes, and compare the PSD of the temporal electrodes and the PSD of the extra-temporal electrodes, wherein the comparison of the temporal electrodes and the extra-temporal electrodes forms a PSD feature integrated with the at least one clinical variable.

19. The system of claim 12, wherein the at least one processor is configured to:
determine a curve from the peri-ictal segment of the EEG data, wherein the curve indicates PSD versus frequency;
determine an area under the curve, within a frequency band; and
determine a proportional area of the frequency band by dividing the area under the curve within the frequency band by a total area under the curve across all frequency bands.

20. The system of claim 12, wherein the EEG system is a non-invasive scalp EEG system, comprises at least 10 temporal electrodes that generate the EEG data, and comprises at least 10 extra-temporal electrodes that generate the EEG data.
